(19) United States
(12) Patent Application Publication
(10) Pub. No.: US 2003/0060825 A1
(43) Pub. Date: Mar. 27, 2003

(54) Anterior lumbar spacer

(76) Inventors: Arthur A. Alfaro, Colts Neck, NJ (US); Lawrence A. Shimp, Morganville, NJ (US); John Boyle, Upper Montclair, NJ (US); Perry Geremakis, Manalapan, NJ (US); James Russell, Little Silver, NJ (US); Nelson L. Scarborough, Andover, MA (US)

Correspondence Address: PETER G. DILWORTH, ESQ., DILWORTH & BARRESE, LLP, 333 Earle Ovington Blvd., Uniondale, NY, 11553, US

(21) Appl. No.: 10/237,821

(22) Filed: Sep. 9, 2002

Related U.S. Application Data
(63) Continuation of application No. PCT/US01/07829, filed on Mar. 9, 2001.
(60) Provisional application No. 60/188,138, filed on Mar. 9, 2000.

(51) Int. Cl. A61B 17/56

(52) U.S. Cl. 606/61

(57) ABSTRACT

There is provided a combined two-part intervertebral implant (10) configured to restore the spacing between adjacent vertebrae. The implant (10) generally includes a spacer ring (12) having upper and lower vertebral engaging surfaces and a bore for receipt of a locking element (14). The implant (10) further includes a locking element (14) which is engageable within the spacer ring (12) and has a diameter or height greater than the thickness of the spacer ring. In one embodiment, the spacer ring (12) may be formed as a C-shaped element (16). In an alternative embodiment, the spacer ring may be formed as an intact ring (42) having a side bore (44) for receipt of the locking element. One or both parts of the implant (10) may be partially or wholly surface demineralized to provide a flexible surface on implant. A method of using the spacer ring (12) and locking element (14) to secure the assembled implant between adjacent vertebrae is also disclosed.

### BACKGROUND OF THE INVENTION

1. Technical Field

The present disclosure relates to an intervertebral implant for spinal fusion and, more particularly, to a two part intervertebral spacer having a large vertebral supporting surface area and structure to lock the spacer within the intervertebral space to prevent expulsion.

2. Background of Related Art

The spine is a flexible column formed of a series of bone called vertebrae. The vertebrae are hollow and piled one upon the other, forming a strong hollow column for support of the cranium and trunk. The hollow core of the spine houses and protects the nerves of the spinal cord. The different vertebrae are connected together by means of articular processes and intervertebral, fibro-cartilages. In general, a vertebral body is made of a cortical shell enclosing a cancellous (spongy) bone core. The portion of the cortical bone shell facing the surface of the disk is the endplate.

The intervertebral fibro-cartilages are also known as intervertebral disks and are made of a fibrous ring filled with pulpy material. The disks function as spinal shock absorbers and also cooperate with synovial joints to facilitate movement and maintain flexibility of the spine. When one or more disks degenerate through trauma, spondylolisthesis or other pathologies, nerves passing near the affected area may be compressed and are consequently irritated. The result may be chronic and/or debilitating back pain. Various methods and apparatus, both surgical and non-surgical, have been designed to relieve such back pain.

One method designed to relieve such back pain is interbody spinal fusion. Typically, interbody spinal fusion involves distracting adjoining vertebrae of the spine so that the nerve root canal sizes are increased and nerve irritation is eliminated or reduced. In order to maintain the adjoining vertebrae in a distracted state, at least one intervertebral implant is inserted into a receiving bed formed between the vertebrae. The implant is positioned to engage the adjoining vertebrae to maintain the vertebrae at a fixed degree of distraction.

Preferably, the implant should stabilize the intervertebral space and become fused to adjacent vertebrae in order to prevent the implant and adjacent vertebrae from moving. The implant must also provide spinal load support between the vertebrae. Further, during the time it takes for fusion, i.e. biological fixation of the vertebrae, to be completed, the implant should have enough structural integrity to maintain the space without substantial degradation or deformation of the implant. The implant should also have sufficient stability to remain in place prior to actual completion of bone ingrowth fusion. The implant should include structure which maintains the implant in position between the vertebrae while bone ingrowth is occurring. To facilitate rapid bone growth, and thus quick fusion, the implant may include or be provided with a bone growth supporting material. Obviously, the material from which the implant is constructed should be a biocompatible material and, preferably, interact biologically with the body's own naturally occurring tissues.

A variety of different types of intervertebral implants have been developed to perform this function including spinal fusion cages, threaded bone dowels and stepped bone dowels. An exemplary implant is disclosed in U.S. patent application Ser. No. 09/328,242, filed on Jun. 8, 1999 and entitled “Ramp-Shaped Intervertebral Implant”, the entire disclosure of which is incorporated by reference herein.

Common deficiencies in some of the prior art implants may include expulsion of the implant from between adjacent vertebrae, difficulty in inserting the implant into position, and/or lack of ability to allow incorporation of implant into the body.

Accordingly, a need exists for an improved intervertebral implant which is configured to prevent the likelihood of expulsion or retropulsion during normal patient activity, provide ease of insertion and include structure to facilitate incorporation of the implant into the body.

### SUMMARY

There is provided a two-part intervertebral spacer for use in restoring the correct disk height between adjacent vertebrae. In one embodiment, the implant includes a generally C-shaped spacer ring having upper and lower vertebral engaging surfaces and an overall pre-determined thickness. The thickness of the ring may vary from a proximal to distal end to provide for a tapered implant. Additionally, the implant includes a locking element which is configured to engage both the spacer ring and adjacent vertebrae to securely lock the implant between adjacent vertebrae. Preferably, the spacer ring has a threaded surface on an inner surface portion of the C-shape and the locking element is a threaded dowel configured to engage the threads in the C-shaped element. The locking element has a height or diameter which is greater than the thickness of the spacer ring such that when the locking element is threaded into the spacer ring and the outer surface of the locking element extends beyond the upper and lower surfaces of the spacer ring so as to engage adjacent vertebral endplates. Preferably, the locking element includes a throughbore for receipt of bone growth inducing materials. Additionally, the locking element may be provided with a bore in its proximal end along with a cross slot for receipt of a suitable insertion instrumentation.

Preferably, one or both of the spacer rings and locking element are formed of a bone material. The inner surface of the C-shaped spacer ring may be threaded prior to insertion or may be threaded after insertion and simultaneously with the formation of threads in the adjacent vertebral endplates. Further, one or both of the spacer ring or locking element may be surface demineralized.

In an alternate embodiment, the spacer ring is formed as an intact circle or ring, preferably having a throughbore through the center thereof. Additionally, a bore is formed in a proximal end of the spacer ring for receipt of a locking element. This bore may be formed prior to or after insertion between the vertebrae. Preferably, the bore of the ring and the locking element are threaded so as to secure the locking element to the spacer ring as well as secure the assembled implant to adjacent vertebrae.

There is also disclosed a method of restoring spacing between adjacent vertebrae which consists of providing a spacer element configured to receive a locking element. The method includes initially displacing or distracting the vertebrae and inserting the spacer ring between the adjacent vertebrae such that the adjacent vertebrae bear on upper and lower surfaces of the ring. The ring may initially be provided with threads in either the inner surface of the C-shaped ring or the bore of the intact ring or may be simultaneously threaded with the formation of threads in adjacent vertebrae. Thereafter the locking element is threaded into the threads formed in the spacer ring and adjacent vertebrae to secure the spacer ring against migration and locking the implant in between the adjacent vertebrae.

### BRIEF DESCRIPTION OF THE DRAWINGS

Various preferred embodiments are described herein with reference to the drawings wherein:

FIG. 1 is a perspective view of an assembled two-part intervertebral spacer;

FIG. 2 is a top plan view of the intervertebral spacer positioned within a disk space;

FIG. 3 is a side perspective view of a first component of the two-part intervertebral spacer positioned between adjacent vertebrae; and

FIG. 4 is a top plan view of an alternate embodiment of a two-part intervertebral spacer positioned within a disk space.

### DETAILED DESCRIPTION OF PREFERRED EMBODIMENTS

Preferred embodiments of the presently disclosed anterior lumbar spacer will now be described in detail with reference to the drawings in which like reference numerals designate identical or corresponding elements in each of the several views. While the preferred use of the spacer is disclosed for use within the lumbar region of the spine, the spacer can be adapted for use within other regions of the spine such as cervical, etc.

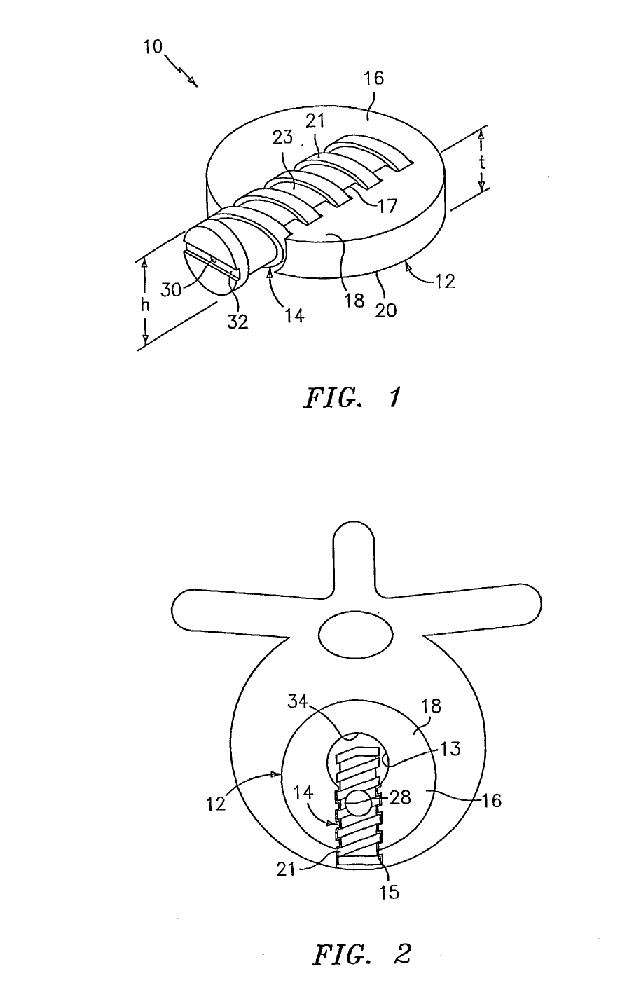

Referring to FIGS. 1 and 2, a two-part intervertebral implant 10 includes a first component or spacer ring 12 and a second component or locking implant 14 configured to prevent migration of implant 10. Spacer ring 12 and/or locking implant 14 can be made from either cortical or cancellous bone or, alternately, from any biocompatible material having the requisite strength requirements including ceramics, polymers, composites, metals such as stainless steel, titanium, etc.

In one embodiment, spacer ring 12 includes a generally C-shaped body 16 having upper and lower vertebral surfaces 18 and 20 defining a thickness “t” therebetween. Surfaces 18 and 20 provided greater vertebral bearing area than a threaded dowel-type implant. Body 16 is dimensioned to fit between intact portions of vertebral end plates 22 and 24 (See FIG. 3) without excessive distraction of the vertebrae. Spacer ring 12 includes an inner surface 13 defining a throughbore 15. In the case of spacer ring being formed of bone, the throughbore 15 may be at least partially a result of the naturally occurring medullary canal. Alternatively, spacer 12 may, prior to insertion, be either a C-shaped ring with the medullary canal or a solid ring, and throughbore 15 formed into the canal or into the entire ring after insertion of body 12 between adjacent vertebrae. Inner surface 13 is preferably provided with threads 17. Upper and lower surfaces 18 and 20 may be partially or wholly surface demineralized to provide a flexible surface that will conform to the contours of vertebral end plates 22 and 24 without the need for machining. By only partial demineralizing body 16, a mineralized core 26 will remain to provide sufficient support to prevent subsidence. The partial or wholly surface demineralization of the surfaces of a bone based implant to form a flexible surface or completely flexible implant is applicable to any bone based implant including weight bearing implants.

Locking implant 14 generally includes a threaded cylindrical dowel having a thread 21 in outer surface 23. Threads 21 may be machine type threads or self-tapping or cutting threads. Locking implant 14 has a diameter or height “h”. The diameter of locking implant 14 may be constant or may vary to form a longitudinal taper. Height h is preferably greater than the thickness t of spacer ring 12. Locking implant 14 may also include a bore 30 and slot 32 at a proximal end thereof for receipt of various installation tools. Locking implant 14 may have a throughbore 28 dimensioned to receive growth factors to stimulate bone growth. The growth factors may include autograft, allograft, DBM, Grafton®, etc.

In use, vertebral endplates 22 and 24 may be distracted and spacer ring 12 positioned between vertebral end plates 22 and 24. Spacer ring 12 should be of proper thickness t to correctly space the vertebrae and may include a ramped or tapered surface (not shown) to provide the proper lordic angle to the vertebrae. Spacer ring may be pre-tapped prior to installation or threaded simultaneously with vertebral surfaces 22 and 24. Thereafter, locking implant 14 is screwed into the adjacent vertebrae and spacer ring 12. Locking implant 14 is preferably threaded until implant 14 engages an inner end surface 34 of ring 12. Alternatively, locking implant 14 may be inserted short of end surface 34.

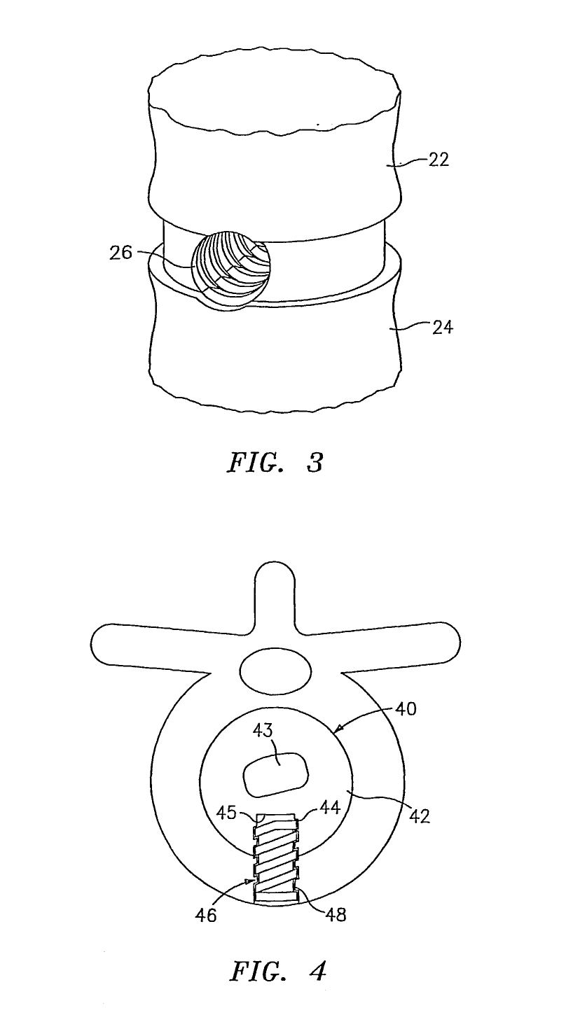

Referring now to FIG. 4, in an alternative embodiment, a two-part implant 40 includes spacer ring 42 which is an intact ring (rather than a C-shaped ring). Spacer ring 42 may be formed from any bio-compatible material and is preferably formed from bone by making transverse cuts across the diaphysis or metaphysics of a long bone, e.g., femur, tiles, tibia, ulna or radius. This process may leave a naturally occurring medullary canal, possibly further treated to remove unwanted material, to form a throughbore 43 for receipt of bone growth factors. Spacer ring 42 is otherwise similar to spacer ring 12 and also has a thickness and upper and lower vertebral engaging surfaces. Spacer ring 42 has a bore 44 for receipt of a locking element. Implant 40 also includes a locking dowel 46 which has threads 48 to engage spacer ring 42 and adjacent vertebrae. Preferably, the diameter or height of locking dowel 46 is greater than the thickness of spacer ring 42. Locking implant 46 need not extend so far as to engage the inner end surface 45 of ring 42, i.e., the distal end of locking implant 46 can be spaced from the inner end surface 45 of ring 42 and/or need not extend to the end 45 of bore 44.

In use spacer ring 42 is positioned between adjacent vertebral end plates to space the vertebrae. Thereafter, ring 42 and the adjacent vertebrae are drilled and tapped to define a threaded receiving bed for receiving locking dowel 46. When locking dowel 46 is inserted between the vertebrae, the dowel threads 48 engage both the spacer ring 42 and the vertebral end plates. This procedure improves fixation of the spacer ring and substantially eliminates migration. Alternately, this procedure may be performed using a spacer ring and/or a locking dowel not formed of bone. However, the spacer ring must be formed of a material that can be machined, i.e., reamed and tapped, in place.

In an alternate method of insertion, bore 44 of the spacer ring 42 is threaded prior to placement between adjacent vertebral end plates. Using this method, it is important that the threaded channel formed between the vertebral end plates correspond substantially with the threads on the spacer ring 42. Thereafter, the spacer ring is positioned between the vertebral end plates, the adjacent vertebrae are reamed and tapped, and the locking dowel 46 is threaded into the spacer ring and adjacent vertebrae.

As discussed above, where spacer rings 12 or 42 are formed of bone, it may be partially demineralized using, for example, a controlled acid treatment, to yield a spacer ring having demineralized upper and lower surfaces which are located to contact the vertebral end plates. The depth of the demineralization of the upper and lower surfaces can be controlled to give a flexible surface layer that will conform to the contours of the vertebral end plates, yet be backed up by strong, mineralized bone to prevent subsidence. The depth of demineralization may be between 0.1 to 2 mm, but is preferably 0.75 to 1 mm. Such demineralized spacer rings may be used alone or in conjunction with the locking ring described above. Moreover, any bone implant can be wholly or partially surface demineralized to allow it to conform to the shape of the bone into which it is being inserted, i.e., this procedure is not limited to implants for intervertebral use. Further, the degree of demineralization may be varied accordingly to provide the bone implant with the appropriate degree of flexibility.

Where spacer ring is formed from a material other than bone, a biocompatible, flexible material, e.g., flexible polymer, may be provided on the weight bearing surfaces of the spacer ring. Such material should have a flexibility sufficient to conform to the shape of the vertebral end plates when the spacer ring is positioned between the vertebral end plates.

When bone is used to form the implants described above, growth factors may be added to the bone to stimulate bone growth and incorporation.

It will be understood that various modifications may be made to the embodiments disclosed herein. For example, either or both of the spacer ring or locking element may be tapered. Additionally, spacer ring need not be circular but can have other shapes such as oval, rectangular, etc. Further, as noted above, the spacer ring may initially be a solid intact disk without any bores and subsequently modified, either before or after insertion, to provide the bores for the locking elements and/or bone growth factors. Therefore, the above description should not be construed as limiting, but merely as exemplification of preferred embodiments. Those skilled in the art will envision other modifications within the scope and spirit of the claims appended hereto.

What is claimed is:

1. A two-part intervertebral spacer comprising:
a first component having upper and lower vertebral engaging surfaces and a thickness between the upper and lower surfaces; and
a second component engagable within the first component and having a height greater than the thickness of the first component.

2. The intervertebral spacer as recited in claim 1, wherein the first component is a C-shaped ring.

3. The intervertebral spacer as recited in claim 2, wherein at least a portion of an inner surface of the ring defined by the C-shape is threaded.

4. The intervertebral spacer as recited in claim 3, wherein the second component is a cylindrical locking element having threads on an outer surface thereof, which threads are engagable with the threads on the inner surface of the C-shaped ring.

5. The intervertebral spacer as recited in claim 1, wherein at least one of the first component and second component is formed from bone.

6. The intervertebral dowel as recited in claim 5, wherein the first component is formed from bone and is partially demineralized to leave a mineralized core of the first component to provide sufficient support to provide subsidence.

7. The intervertebral spacer as recited in claim 5, wherein at least one of the upper and lower vertebral engaging surfaces are wholly or partially surface demineralized to provide a flexible surface to conform to adjacent vertebral endplates.

8. The intervertebral implant as recited in claim 4, wherein the locking element includes a throughbore for receipt of bone growth inducing factors.

9. The intervertebral implant as recited in claim 1, wherein the first component is an intact ring having a bore in an outer surface thereof and the second component is a dowel configured to engage an inner surface of the bore.

10. The intervertebral implant as recited in claim 9, wherein the outer surface of the dowel and the inner surface of the bore are formed with corresponding mating threads.

11. The intervertebral spacer as recited in claim 9, wherein the intact ring defines a throughbore for receipt of bone growth inducing factors.

12. A two-part intervertebral spacer comprising:
a generally C-shaped ring defining a throughbore and having a predetermined thickness between an upper and a lower vertebral engaging surface;
a threaded dowel having a diameter greater than the predetermined thickness and engagable within the C-shaped ring.

13. The intervertebral spacer as recited in claim 12, wherein at least one of the C-shaped ring and threaded dowel are formed of bone.

14. The intervertebral dowel as recited in claim 13, wherein the C-shaped ring is formed of bone and at least one of the upper and lower vertebral surface is at least partially surface demineralized.

15. The intervertebral spacer as recited in claim 12, wherein an inner surface of the C-shaped ring which defines the throughbore is formed with threads.

16. The intervertebral spacer as recited in claim 12, wherein the threaded dowel defines a throughbore for receipt of bone growth inducing factors.

17. The intervertebral spacer as recited in claim 12, wherein the threaded dowel includes structure for receipt of insertion instrumentation.

18. A method of restoring spacing between adjacent vertebrae comprising:
providing a two-part intervertebral spacer having a ring defining a bore and upper and lower vertebral engaging surfaces defining a thickness between the upper and lower surfaces and a locking implant engagable within the bore of the ring and having a height greater than the thickness of the ring;
positioning the ring within an excised disk space between adjacent vertebrae; and
engaging the locking implant within the ring and with the adjacent vertebrae.

19. The method according to claim 18 wherein the step of engaging includes threadedly engaging threads formed on an inner surface of the bore with threads formed on an outer surface of the locking implant.

20. The method according to claim 18 wherein, prior to the step of engaging, threads are simultaneously formed on an inner surface of the bore and at least one endplate of adjacent vertebrae.

21. The method of claim 18, wherein the bore defined in the ring is formed after the step of positioning the ring.
